(19) United States
(12) Patent Application Publication
(10) Pub. No.: US 2017/0267546 A1
(43) Pub. Date: Sep. 21, 2017

(54) Method of Monitoring Naphthenic Acids

(71) Applicant: Ecolab USA Inc., St. Paul, MN (US)

(72) Inventors: Neli Enache, Sarnia (CA); James Walker, Camlachie (CA); Brendan Kranzmann, Streamwood, IL (US); David Dvorchak, Naperville, IL (US); Thomas Archakis, Loudon, TN (US); Michael Murcia, DeKalb, IL (US); Andrew Eisenhawer, Sherwood Park (CA)

(73) Assignee: Ecolab USA Inc., St. Paul, MN (US)

(21) Appl. No.: 15/460,809

(22) Filed: Mar. 16, 2017

Related U.S. Application Data
(60) Provisional application No. 62/309,233, filed on Mar. 16, 2016.

(51) Int. Cl. C02F 1/00; G01N 21/64; G01N 33/18; C02F 1/28

(52) U.S. Cl. CPC C02F 1/008; C02F 1/004; C02F 1/283; G01N 21/645; G01N 33/1833; C02F 2101/32

(57) ABSTRACT

Disclosed are methods of monitoring the presence of naphthenic acids and related compounds. In particular, the invention provides a method of continuously monitoring naphthenic acids and related compounds that break through a filtration step in a wastewater treatment process.

This application is an international (i.e., PCT) application claiming the benefit of U.S. Provisional Patent Application No. 62/309,233, filed Mar. 16, 2016, the contents of which are incorporated by reference herein in their entirety.

### FIELD OF INVENTION

The invention is directed to a method of monitoring naphthenic acids and related species in a wastewater treatment process using fluorescence-based detection.

### BACKGROUND OF THE INVENTION

The petroleum industry utilizes water for a variety of physical and chemical treatment processes. Crude oil refineries generate a large amount of wastewater from systems and processes including desalting processes, hydrotreating, distillation, and cooling systems, while extraction and processing of petroleum from oils sands likewise requires large volumes of caustic water for recovery of bitumen from sand and clay. The wastewater obtained from such processes often contains high levels of chemical pollutants that can pose serious risk to the environment.

Naphthenic acids are naturally-occurring compounds commonly found in crude oil and are thought to originate from aerobic microbial degradation of petroleum hydrocarbons. Naphthenic acids are a complex mixture of hydrocarbon compounds of varying structure, and typically range from a molecular weight of 120 to over 1,300 Daltons. The term “naphthenic acids” is often applied to all acidic water-extractable components of hydrocarbons, which, in addition to true naphthenic acids, typically comprise compounds having various levels of unsaturation and aromaticity, including compounds such as phenols, pyrroles, and thiophenes.

Process wastewater generated by refining and oil recovery activities typically undergoes water reclamation or is released into the environment. Naphthenic acids are known environmental toxicants, and can affect the existence, growth, and proliferation of aquatic organisms such as fish and plants, as well as invertebrates. The natural biodegradability of naphthenic acids is generally low. Water treatment strategies must employ an acceptable means of purifying process water containing naphthenic acids prior to environmental release.

Furthermore, water originating from various unit processes is often reclaimed and recycled back into the refinery or oil extraction production as process supply water. For example, water is recycled back to the process plant as part of a “zero discharge” policy followed by many oil sand extraction companies. Naphthenic acids can be removed from recycled water using an appropriate water treatment strategy to minimize downstream impacts such as fouling of boilers.

To determine the effectiveness of the removal of naphthenic acids from process effluent, wastewater treatment strategies typically include an analytical method for determining the efficacy and efficiency of the employed purification method. The complexity of naphthenic acid mixtures complicates the development of suitable analytical methods for monitoring and detecting naphthenic acid levels in purified wastewater. There are a number of analytical methods available, but many are either ineffective or are difficult to perform in a continuous method.

Accordingly, there is a need for a method of monitoring the purification of process-generated wastewater. The present methods provide an accurate measurement of naphthenic acids in wastewater and can be used to determine if the wastewater is suitable for reclamation or release into the environment.

### BRIEF SUMMARY OF THE INVENTION

In an embodiment, the invention provides a method of monitoring naphthenic acids in wastewater. The method comprises filtering wastewater comprising naphthenic acids through a filter capable of removing naphthenic acids from the wastewater, and contacting the filtered wastewater with a fluorescence sensor capable of detecting chromophoric components of naphthenic acids and detecting the presence of naphthenic acids in the filtered wastewater.

In another embodiment, the invention provides a method of monitoring naphthenic acids in wastewater. The method comprises filtering wastewater comprising naphthenic acids through a filter having a fluorescence sensor embedded therein and capable of removing naphthenic acids from the wastewater, and contacting the filtered wastewater with a fluorescence sensor capable of detecting chromophoric components of naphthenic acids and detecting the presence of naphthenic acids in the filtered wastewater.

In another embodiment, the invention provides a method of monitoring naphthenic acids in wastewater. The method comprises filtering wastewater comprising naphthenic acids through a filter capable of removing naphthenic acids from the wastewater, and contacting the filtered wastewater with a fluorescence sensor capable of measuring the light absorbance or transmittance of naphthenic acids in the filtered wastewater.

These and other features and advantages of the present invention will be apparent from the following detailed description, in conjunction with the appended claims.

### BRIEF DESCRIPTION OF THE DRAWINGS

FIG. 1 is a calibration curve displaying fluorescence intensity vs. concentration of total naphthenic acids in a wastewater sample.

FIG. 2 is a calibration curve displaying fluorescence intensity vs. concentration of total naphthenic acids in a wastewater sample.

FIG. 3 is a calibration curve displaying UV transmittance vs. concentration of total naphthenic acids in a wastewater sample.

FIG. 4 is a calibration curve displaying UV transmittance vs. concentration of total naphthenic acids in a wastewater sample.

### DETAILED DESCRIPTION OF THE INVENTION

The following definitions are provided to determine how terms used in this application, and in particular, how the claims are to be construed. The organization of the definitions is for convenience only and is not intended to limit any of the definitions to any particular category.

“Fluorescence sensor” as used here, refers to a device having a light detector and optionally, an integrated excitation light source. The light detector may be capable of filtering excitation wavelength(s) of light from emission wavelength(s) of light using, e.g., a monochromatic filter, dichroic filter, or long pass filter;

“Naphthenic acids” as used here, refers to a complex mixture of aliphatic and aromatic carboxylic acids, phenols, glycols and other polar hydrocarbon components extractable into water from crude oil or crude oil distillation fractions. Naphthenic acids typically include chromophoric components. Naphthenic acids have varying degrees of fluorescence and/or toxicity when dispersed or dissolved in water; the degree of either may depend on environmental conditions such as pH, salinity and temperature. The term naphthenic acids may also refer to naphthenates or naphthenate salts;

“Monitoring” means any type of tracing or tracking to determine the presence, amount, or concentration of naphthenic acid at any given site, including singular, intermittent, or continuous monitoring;

“Wastewater” means water from a manufacturing process, such as oil refining or extraction, that is required to be treated prior to discharge to a receiving stream, lake, or other water way. Those having skill in the art will recognize that the disclosure refers to raw wastewater as any aqueous fluid that without prior treatment is not suitable for environmental release or industry application or discharge from any facility because of the existence of natural or artificial contaminants. Nonlimiting examples of contaminants include organics, particulates, and sub-micron particles.

Methods are provided that can be used to effectively monitor the filtration of wastewater. More particularly, methods are provided that can be used to monitor naphthenic acids in filtered wastewater using fluorescence measuring. Monitoring and quantification of naphthenic acids in water has traditionally been performed by extraction of filtered water with organic solvents and analysis of naphthenic acids in the resulting organic solution, which is costly and inconvenient for a continuous monitoring method. Applicants have discovered that a system of filtering wastewater and directly monitoring naphthenic acids in the filtered wastewater provides an effective and efficient method of monitoring the effectiveness of a wastewater filtration process. Applicants have further discovered that the combination of wastewater filtration and fluorescence monitoring can maintain naphthenic acids in filtered wastewater at a concentration lower than 5 ppm. The present methods allow for immediate detection of breakthrough of naphthenic acids through a filter, and can be used to automatically alert an operator when such filter breakthrough occurs. The present invention also provides a method of continuously monitoring the concentration of naphthenic acids in a filtered wastewater.

Naphthenic acids are a complex mixture of compounds that typically comprise both aliphatic and aromatic compounds. The mixture of compounds, which are natural components of petroleum, can be removed using a filtration process. In certain embodiments, wastewater is contacted with a filter. In certain embodiments, the filter comprises an adsorbent selected from the group consisting of carbon, zeolite, clays such as kaolin and/or bentonite, and combinations thereof. In certain preferred embodiments, wastewater is contacted with a filter comprising activated carbon. As wastewater moves through the filter, naphthenic acids will transfer from the wastewater to the filtration material. In particular, when the filter comprises activated carbon, it is believed that naphthenic acids are removed from wastewater by mass transfer of the naphthenic acids to the surface of the carbon particles, diffusion of the naphthenic acids through carbon pores, and adsorption of the naphthenic acids to the surface of the carbon particles. As more wastewater passes through the filter, the adsorbed naphthenic acids move toward the end of the filter. If the filter is saturated with the adsorbed naphthenic acids, a breakthrough of naphthenic acids can result because the filter no longer has the ability to remove naphthenic acids from the wastewater. Breakthrough of naphthenic acids may also occur due to wear, age, or puncture of the filter. Facile and accurate detection of a breakthrough event is important to the wastewater purification process. In certain embodiments, a filtration process is continuously monitored to enable immediate identification of a breakthrough event so that the filter can be refilled, regenerated, or replaced.

Naphthenic acids are known to contain impurities that include various levels of unsaturation and aromaticity (e.g., aromatic acids and phenols). The chromophoric components present in naphthenic acids can serve as an internal standard for indirect analysis of total naphthenic acids content. The chromophoric components of naphthenic acids can give rise to fluorescent signals when excited by light at a particular wavelength, which can be detected using a fluorescence emission detector. In an embodiment, the present invention involves monitoring filtered wastewater by fluorescent measurement of chromophoric components of naphthenic acids in the filtered wastewater. Fluorescence monitoring correlates fluorescence to the concentration of naphthenic acids in filtered wastewater, which allows for immediate detection and measurement of naphthenic acid breakthrough. The present invention can be used to continuously monitor the concentration of naphthenic acids in filtered wastewater.

In an embodiment, the invention provides a method of monitoring naphthenic acids in wastewater. The method comprises filtering wastewater comprising naphthenic acids through a filter capable of removing naphthenic acids from the wastewater, and contacting the filtered wastewater with a fluorescence sensor capable of detecting chromophoric components of naphthenic acids and detecting the presence of naphthenic acids in the filtered wastewater.

In certain preferred embodiments, the fluorescence of the filtered wastewater is measured. In certain preferred embodiments, the measured fluorescence is correlated with the concentration of naphthenic acids in the filtered wastewater using a calibration curve. A calibration curve may be created by plotting the measured fluorescence of the wastewater versus the concentration of naphthenic acids (e.g., total naphthenic acids, chromophoric components of naphthenic acids, non-chromophoric components of naphthenic acids). Thus, in certain embodiments, a calibration curve is used to determine the concentration of chromophoric components of naphthenic acids, non-chromophoric components of naphthenic acids, and/or total naphthenic acids.

In certain embodiments, the wastewater is filtered through a filter comprising or consisting of a single filter unit. In certain embodiments, the wastewater is filtered through a filter comprising or consisting of two filters operating in parallel. In certain embodiments, the wastewater is filtered through a filter comprising or consisting of two filters operating in series. In certain embodiments, the wastewater is filtered through a filter comprising or consisting of three or more filters operating in parallel. In certain embodiments, the wastewater is filtered through a filter comprising or consisting of three or more filters operating in series. In certain embodiments, the wastewater is filtered through multiple groups of filters (i.e., trains) in series, with the trains operating in parallel.

In certain other embodiments, the present invention is used to monitor wastewater from a filtration process that utilizes fixed-bed adsorption, expanded-bed adsorption, moving-bed adsorption, fluidized bed adsorption, or combinations thereof. In certain preferred embodiments, the filtration occurs via a downflow fixed-bed adsorber process. However, the present invention can be used to monitor upflow wastewater as well.

In certain embodiments, a fluorescence sensor is present or positioned inside a filter or adsorption column. In certain embodiments, the fluorescence sensor is present or positioned between filters or adsorption columns. In certain embodiments, the fluorescence sensor is present or positioned between each filter or adsorption column. In certain preferred embodiments, the fluorescence sensor is present or positioned at an effluent outlet of the filter or adsorption column. In certain embodiments, the fluorescence sensor is present or positioned at the effluent outlet of the first filter or adsorption column. In certain embodiments, the fluorescence sensor is present or positioned at the effluent outlet of each filter or adsorption column.

The fluorescence sensor is contacted with the filtered wastewater. In certain embodiments, the fluorescence sensor monitors the concentration of naphthenic acids in the filtered wastewater by illuminating a certain wavelength(s) of light and detecting the light emitted by naphthenic acids in the filtered wastewater. The excitation wavelengths and emission wavelengths monitored can be chosen in order to balance selectivity, sensitivity, and signal-to-noise ratio.

Any suitable excitation wavelength can be used to excite the naphthenic acid molecules present in wastewater. In certain embodiments, filtered wastewater is exposed to an excitation wavelength of from about 200 nm to about 900 nm. In certain embodiments, filtered wastewater is exposed to an excitation wavelength of from about 200 nm to about 500 nm. Thus, in certain embodiments, wastewater is exposed to an excitation wavelength of from about 200 nm to about 500 nm, from about 200 nm to about 450 nm, from about 200 nm to about 400 nm, from about 200 nm to about 350 nm, from about 200 nm to about 300 nm, from about 250 nm to about 500 nm, from about 250 nm to about 450 nm, from about 250 nm to about 400 nm, from about 300 nm to about 500 nm, or from about 300 nm to about 450 nm. In certain preferred embodiments, filtered wastewater is exposed to an excitation wavelength of from about 220 nm to about 300 nm. Thus, in certain embodiments, wastewater is exposed to an excitation wavelength of from about 220 nm to about 300 nm, from about 220 nm to about 290 nm, from about 220 nm to about 280 nm, from about 220 nm to about 270 nm, from about 220 nm to about 260 nm, from about 220 nm to about 250 nm, from about 230 nm to about 300 nm, from about 240 nm to about 300 nm, from about 250 nm to about 300 nm, from about 260 nm to about 300 nm, from about 220 nm to about 250 nm, or from about 260 nm to about 275 nm. In certain embodiments, an excitation wavelength of from about 220 nm to about 250 nm excites certain naphthenic acids to a greater extent than certain phenols. In certain embodiments, an excitation wavelength of from about 260 nm to about 275 nm excites certain naphthenic acids to a greater extent than certain phenols.

The fluorescent emission of the filtered wastewater can be monitored or measured at any suitable wavelength. In certain embodiments, the fluorescent emission of the filtered wastewater is monitored or measured at a wavelength of from about 300 nm to about 900 nm. In certain embodiments, the fluorescent emission of filtered wastewater is monitored or measured at a wavelength of from about 300 nm to about 600 nm. Thus, in certain embodiments, the fluorescent emission of filtered wastewater is monitored or measured at a wavelength of from about 300 nm to about 600 nm, from about 300 nm to about 550 nm, from about 300 nm to about 500 nm, from about 350 nm to about 600 nm, from about 400 nm to about 600 nm, or from about 350 nm to about 550 nm.

The wastewater contacts the fluorescence sensor at any suitable temperature. In certain preferred embodiments, the wastewater is at a temperature of about 1° C. or more when the wastewater contacts the fluorescence sensor. In certain preferred embodiments, the wastewater is at a temperature of from about 20° C. to about 100° C. when the wastewater contacts the fluorescence sensor. In certain embodiments, the wastewater is at a temperature of from about 20° C. to about 50° C. when the wastewater contacts the fluorescence sensor. In certain preferred embodiments, the wastewater is at a temperature of about 25° C. when the wastewater contacts the fluorescence sensor.

In certain preferred embodiments, fluorescence measurement of the wastewater that exits from the filter is used to determine whether the filter requires replacement or a change in at least one parameter (e.g., particle size, height of absorption bed, column diameter). In certain preferred embodiments, the fluorescence measurement of the wastewater that flows through a first filter is used to determine if a second filter requires replacement or change in at least one parameter (e.g., particle size, height of absorption bed, column diameter) to remove any naphthenic acids resulting from a breakthrough. Thus, in certain embodiments, the present method is used to limit or control breakthrough of naphthenic acids through a first filter. In addition, in certain embodiments, the present method is used to determine when an increase in filtration load or if an additional filter is required.

In certain embodiments, a threshold value is used to identify if the filtered wastewater comprises naphthenic acids exceeding a desired concentration. The predetermined threshold can be of any concentration. As understood by those skilled in the art, the predetermined threshold will depend on the targeted wastewater strategy (e.g., reclamation or disposal). If the concentration of naphthenic acids is below the predetermined threshold, the filtered wastewater may be released into the water shed or reused in an industrial process (e.g., oil extraction or oil refinery). If the concentration of naphthenic acids is above the predetermined threshold, the filter may need to be replaced or the parameters of the filter altered to obtain optimal filter performance. In certain preferred embodiments, the filter is replaced if the concentration of the naphthenic acid in filtered wastewater is greater than the predetermined concentration threshold.

The predetermined threshold as defined by the user can be any desired concentration. In certain embodiments, the predetermined threshold is a concentration of about 1 ppm or more. In certain embodiments, the predetermined threshold is a concentration of about 5 ppm or more. In certain embodiments, the predetermined threshold is a concentration of about 10 ppm or more. The predetermined threshold can be either the amount of chromophoric components, non-chromophoric components, or total naphthenic acids. In certain preferred embodiments, the predetermined threshold relates to the amount of total naphthenic acids.

In certain embodiments, a fluorescence sensor is connected to a computer that is programmed by the user. The user can program the computer to define the desired upper and lower limits of detection. In certain embodiments, the computer is connected to an alarm or alert system, or comprises an alarm or alert system. The alarm or alert system is automatically initiated by the computer when the fluorescence reading is outside of the defined limits. In certain embodiments, the alert or alarm causes the computer to perform an action, such as feed more chemical, light up a light or sound a siren, or open/close valves. The alarm or alert can also be relayed to a distributed control system (DCS) so that the plant operators get immediate notification of the alarm in their control room display. Additionally, if the computer is connected to a cellular modem, an alarm message can be sent to defined users by phone, email, or text message.

In certain embodiments, an alert or alarm automatically sounds when the filter begins to release wastewater having a concentration of naphthenic acids higher than the desired concentration value or predetermined threshold. In certain preferred embodiments, an alert or alarm automatically sounds when the filter begins to release wastewater having a concentration of naphthenic acids of from about 1% to about 10% above the desired concentration value. In certain preferred embodiments, an alert or alarm automatically sounds when the filter begins to release wastewater having a concentration of naphthenic acid of from about 5% to about 10% above the desired concentration value.

The filter of the present invention can comprise any material. In certain embodiments, the filter comprises, consists of, or consists essentially of an adsorbent selected from the group consisting of carbon, zeolite, clays such as kaolin and/or bentonite, and combinations thereof. In certain preferred embodiments, the filter comprises, consists of, or consists essentially of activated carbon. The activated carbon can be in any available form, such as for example, granules, extrudates, pellets, and combinations thereof. Carbon of any particle size can be used. In certain preferred embodiments, granulated activated carbon has a particle size of about 8×16 mesh to about 20×50 mesh is used. In certain embodiments, pelleted activated carbon having a diameter of from about 0.9 to about 2 mm diameter and from about 3 to about 4 mm length. In certain preferred embodiments, the filters all comprise the same filtration material. In certain embodiments, certain filters comprise a different filtration material than the other filters. In certain embodiments, each filter comprises a different filtration material.

The present invention may comprise a filter having any amount of carbon per volume of wastewater necessary to purify the wastewater. In preferred embodiments, the filter comprises from about 25 to about 200 grams of carbon/m³ filtered wastewater. Thus, in certain embodiments, the filter comprises from about 25 to about 40, from about 25 to about 50, from about 25 to about 75, from about 25 to about 100, from about 25 to about 125, from about 25 to about 150, from about 25 to about 175, from about 25 to about 200, from about 50 to about 200, from about 75 to about 200, from about 100 to about 200, from about 150 to about 200, from about 50 to about 150, from about 50 to about 100, from about 75 to about 150, or from about 75 to about 125 grams of carbon/m³ filtered wastewater.

The present method of detection of filter bleed-through is beneficial because fluorescence measurement provides the sensitivity required to measure concentrations in the range relevant to the filtration process. Furthermore, online fluorometer technology is portable and typically more industrially robust than other spectroscopic technologies.

Fluorometric analysis is generally conducted using a light source and a fluorescence detector (e.g., fluorometer) configured to detect fluorescence as known in the art. The fluorometer is commonly chosen from a filter fluorometer or a spectrofluorometer. In a certain preferred embodiment, the fluorometric techniques are carried out using an excitation light source capable of shining light at a particular wavelength or wavelength range, such as an arc lamp (e.g., mercury, xenon, tungsten-halogen, or xenon-mercury arc lamp), a laser, or a light emitting diode (LED). In certain preferred embodiments, the excitation light source is a light emitting diode (LED).

Crosstalk between the excitation light source and the detector from scattering in the sample due to e.g., turbidity, can be suppressed by maximizing the distance in stokes shift between the excitation and emission filters and by increasing the sharpness of cutoff in the filters. The larger the stokes shift (difference between the excitation wavelength and emission wavelength being monitored), the less chance there is for the excitation light scattered by turbidity to pass through the emission filter and be measured as fluorescence. The sharper the emission filter cutoff is on the blue edge of the filter, the less scattered light gets through. Depending on the potential for turbidity in the water, these parameters are optimized to keep crosstalk at a minimum.

In certain embodiments, it has been discovered that monitoring of fluorescence emission at a wavelength of from about 300 nm to about 400 nm is optimal. In certain embodiments, it has been discovered that monitoring of fluorescence emission at a wavelength of from about 325 nm to about 355 nm is optimal. For example, in certain embodiments, Applicants have discovered that monitoring at a wavelength range of from about 325 nm to about 355 nm adequately excludes scattering crosstalk between the LED and the detector of the fluorometer. This wavelength range is especially useful for situations where turbidity is a factor, giving more accurate results for turbid solutions. Thus, in certain preferred embodiments, filtered wastewater is monitored at a wavelength of from about 325 nm to about 355 nm, from about 325 nm to about 350 nm, from about 325 nm to about 345 nm, from about 325 nm to about 340 nm, from about 325 nm to about 335 nm, from about 325 nm to about 330 nm, from about 330 nm to about 355 nm, from about 335 nm to about 355 nm, from about 340 nm to about 355 nm, from about 345 nm to about 355 nm, from about 330 nm to about 340 nm, or from about 330 nm to about 345 nm.

Another point to consider is that once turbidity in the sample is present, it also scatters the emitted fluorescence light, preventing it from reaching the detector. Therefore, in certain embodiments, the turbidity of the sample is low not only for limiting crosstalk between the excitation light source and detector, but for preventing attenuation of the fluorescence signal.

The present invention can be used to monitor or detect naphthenic acids in wastewater of any turbidity. However, in certain embodiments, the present invention is suitable for monitoring and detecting naphthenic acids in filtered wastewater having a turbidity of about 20 NTU or less. Thus, in certain embodiments, the present invention is suitable for monitoring and detecting naphthenic acids in filtered wastewater having a turbidity of about 20 NTU or less, about 15 NTU or less, about 10 NTU or less, about 5 NTU or less, about 4 NTU or less, about 3 NTU or less, about 2 NTU or less, about 1 NTU or less, or about 0.1 NTU or less. In certain embodiments, the present invention is suitable for monitoring and detecting naphthenic acids in filtered wastewater having no turbidity or essentially no turbidity.

The methods of the present invention can be used to monitor wastewater having any pH. In certain preferred embodiments, the wastewater has a pH of from about 2 to about 12. Thus, in certain preferred embodiments, the wastewater has a pH of from about 2 to about 12, from about 2 to about 11, from about 2 to about 10, from about 2 to about 9, from about 2 to about 8, from about 2 to about 7, from about 6 to about 12, from about 6 to about 11, from about 6 to about 10, from about 6 to about 9, from about 7 to about 12, from about 7 to about 11, from about 7 to about 10, from about 8 to about 12, from about 8 to about 12, from about 9 to about 12, from about 6 to about 10, or from about 5 to about 8. In certain embodiments, the wastewater comprises basic wastewater. In certain embodiments, the wastewater comprises acidic wastewater.

In certain preferred embodiments, the concentration of naphthenic acids in filtered wastewater is monitored continuously.

The present methods can be used to monitor filtration during any wastewater treatment step. In certain embodiments, the present methods are used to monitor filtration after the wastewater has been processed in a biological treatment system. In certain embodiments, the present methods are used to monitor a filtration during tertiary treatment. The monitoring of the filtration can occur in conjunction with other tertiary treatments including sand filtration, chemical oxidation, or combinations thereof.

It should be understood that the present invention is useful for monitoring of a wide variety of wastewater, including, for example, those generated by the petroleum industry. For example, the present method can be used to monitor filtration of wastewater derived from cooling systems, distillation, hydrotreating, desalting, tank drains, equipment flushing, surface water runoff, and sanitary wastewaters associated with the petroleum industry. In general, the present invention has utility for monitoring of any wastewater having naphthenic acids or related compounds. In certain embodiments, the purified wastewater is either reused in the industrial process or released into the environment (e.g., pond, water stream, or water shed). In certain embodiments, the wastewater is a wastewater stream.

In another embodiment, the invention provides a method of monitoring naphthenic acids in wastewater. The method comprises filtering wastewater comprising naphthenic acids through a filter having a fluorescence sensor embedded therein and capable of removing naphthenic acids from the wastewater, and contacting the filtered wastewater with a fluorescence sensor capable of detecting chromophoric components of naphthenic acids and detecting the presence of naphthenic acids in the filtered wastewater.

In certain embodiments, the filter is capable of measuring fluorescence at more than one location in the filter. The advantages of doing so are (a) improved prediction of when to change the filtration material, (b) it provides an absorption rate between the points, and (c) the ability to detect a wave of material that passes deeper into the filter before being absorbed. In certain embodiments, a warning alarm may alert the operator that breakthrough may occur before the filter is exhausted. This enables an operator to replace or alter the filter before breakthrough occurs.

Moreover, the fluorescence intensity can be separately calibrated for each location to the concentration of the species of concern. For example, in a refinery effluent, the presence of toxic organics such as naphthenic acids can be removed by activated carbon filtration. In the present invention, the concentration of these compounds, and to some extent, the toxicity of the effluent, can be correlated with the fluorescence intensity at various points in the flow path such as at the inlet, interior, or outlet of the filter. In certain embodiments, the sensor is embedded by connecting the sensor to the filter inlet or filter outlet, or a port which connects at least two filters.

In another embodiment, the invention provides a method of monitoring naphthenic acids in wastewater. The method comprises filtering wastewater comprising naphthenic acids through a filter capable of removing naphthenic acids from the wastewater, and contacting the filtered wastewater with a fluorescence sensor capable of measuring the light absorbance or transmittance of naphthenic acids in the filtered wastewater.

In certain preferred embodiments, the light absorbance or transmittance of naphthenic acids in filtered wastewater is measured. In certain preferred embodiments, the absorbance or transmittance is correlated with the concentration of naphthenic acids in the filtered wastewater using a calibration curve. A calibration curve may be created by plotting the measured light absorbance or transmittance of the wastewater versus the concentration of naphthenic acids (e.g., total naphthenic acids, chromophoric components of naphthenic acids, non-chromophoric components of naphthenic acids). In certain preferred embodiments, the UV light absorbance or transmittance of the filtered wastewater is measured using a spectrophotometer or the like.

Those skilled in the art will appreciate that the present invention may employ treatment chemicals and other treatment methods. Multiple treatments can be used separately or together. For example, the present invention can be used to monitor filtration in combination with other treatments, such as desalter effluent pretreatment, secondary treatments such as suspended growth process and attached growth processes, chemical oxidation, biological treatment, sludge treatments, water segregation, oil/water separators, or combinations thereof.

In certain embodiments, the invention provides the ability to monitor and control the replacement of a filter in real time using TRASAR or 3D TRASAR technology, or a similar technology. The ability to automate such treatment can improve the efficiency and reduce total cost of operation of purification of wastewater treatment systems. The invention at hand can be used to improve effluent quality for regulatory compliance and system stability.

The present invention can be used to monitor any filtered wastewater that may comprise naphthenic acids. In certain preferred embodiments, the present invention is used to monitor wastewater discharged from a petroleum process. In certain preferred embodiments, the present invention is used to monitor wastewater discharged from an oil refinery. In certain preferred embodiments, the present invention is used to monitor wastewater discharged from an oil extraction process, such as extraction of petroleum from oil sands.

The following examples further illustrate the invention but, of course, should not be construed as in any way limiting its scope.

### Example 1

This Example illustrates the construction and linearity of a calibration curve for total naphthenic acids present in oil process effluent. The calibration curve plots the fluorescence intensity vs. known concentration of total naphthenic acids.

General Chemistry Methods. Samples from various steps in the wastewater process were collected. The concentration of total naphthenic acids in process effluent samples obtained from various steps of the oil process was determined. Each sample was filtered through a 0.45 micron PTFE filter. Fluorometric analysis was performed on a Nalco 3D TRASAR fluorometer fitted with an ultraviolet diode. In the absence of light, about 3 ml of each sample was injected into the 3D TRASAR fluorometer and the resulting fluorescence was recorded. The samples were subjected to a fluorescence excitation wavelength of 280 nm. The fluorescence emission of the wastewater samples was monitored using a photodiode over an average fluorescence emission wavelength of 327 nm to 353 nm. It was discovered that the chosen fluorescence emission wavelength resulted in exclusion of scattering crosstalk between the LED and the detector, especially for turbid water samples.

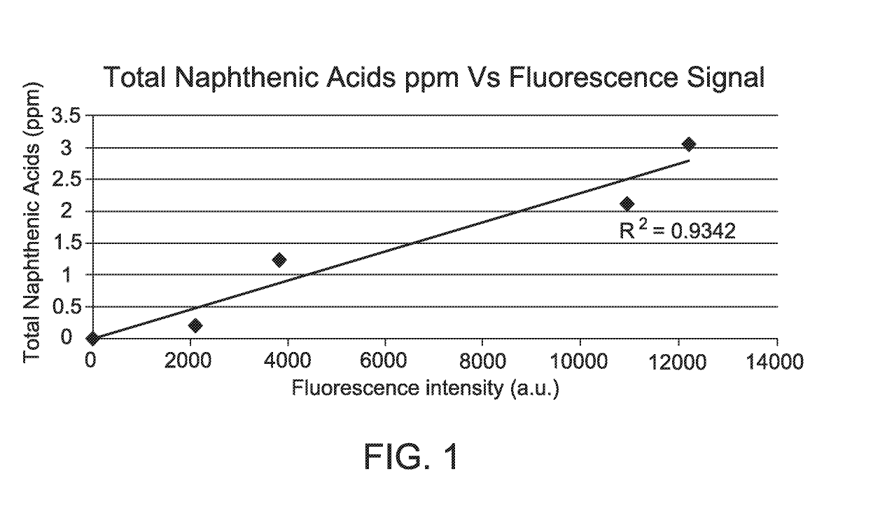

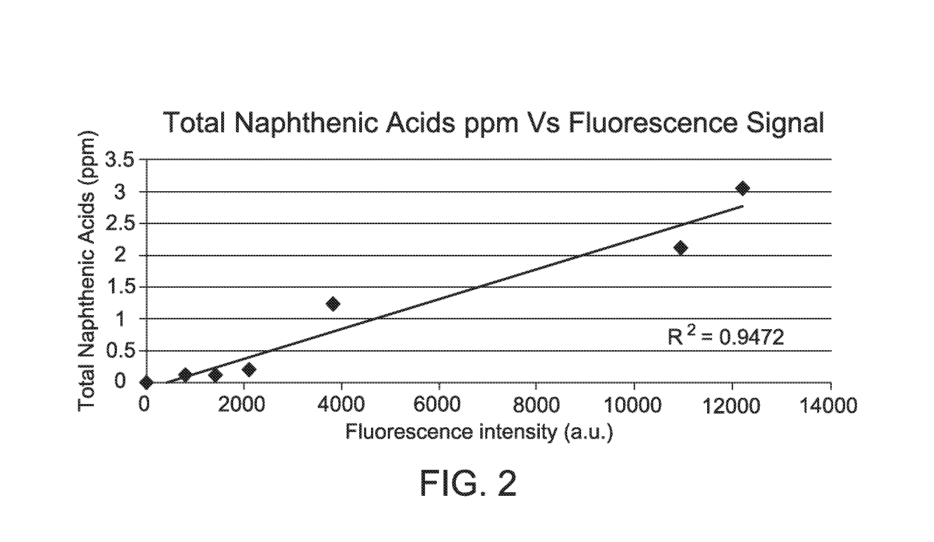

Calibration curves are shown for naphthenic acid from oil process effluent in FIGS. 1 and 2, where fluorescence intensity is plotted along the horizontal axis and the total naphthenic acids concentration is plotted along the vertical axis.

The relationship between the two parameters is clear, as the calibration of total naphthenic acids has good linearity (R² up to 0.95). The slight deviation from linearity may be due to differing species of naphthenic acids found at the various sample points in the process, which would have different responses to the lab ppm analysis and fluorescence readings. It is believed that if the samples were pulled from a single point in the process, even greater linearity would be observed.

### Example 2

This Example illustrates the construction and linearity of a calibration curve for total naphthenic acids in oil process effluent, which plots the UV transmittance vs. known concentration of total naphthenic acids.

The samples from various points in the wastewater process were collected and filtered through a 0.45 micron PTFE filter. In the absence of light, about 3 ml of each sample was injected into a 3D TRASAR fluorometer and the resulting fluorescence and transmittance were recorded. The data was collected using the same 3D sensor as used for Example 1 on a photodiode located 180 degrees across the flow cell from the LED.

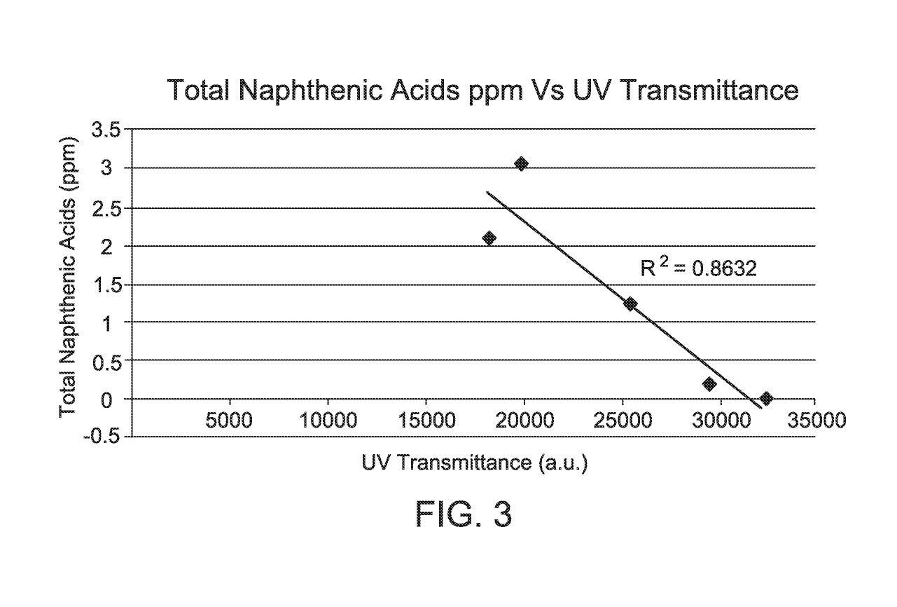

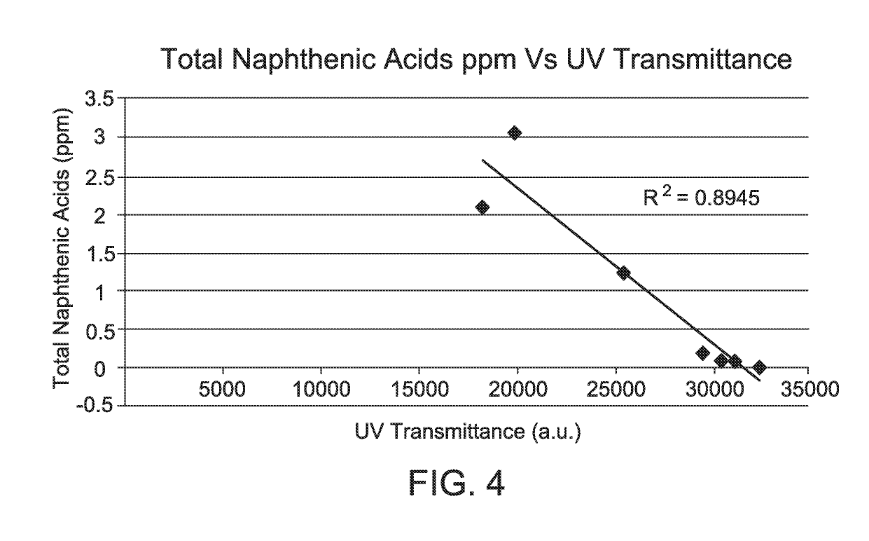

Calibration curves are shown for total naphthenic acids from oil process effluent in FIGS. 3 and 4, where UV transmittance is plotted along the horizontal axis and the total naphthenic acids concentration is plotted along the vertical axis. Overall, the calibration of total naphthenic acids has good linearity (R² up to 0.89).

This Example demonstrates that UV transmittance can be used to monitor wastewater to determine concentration of naphthenic acids in wastewater in accordance with an embodiment of the present invention.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A method of monitoring naphthenic acids in wastewater, the method comprising:
filtering wastewater comprising naphthenic acids through a filter capable of removing naphthenic acids from the wastewater; and
contacting the filtered wastewater with a fluorescence sensor capable of detecting chromophoric components of naphthenic acids and detecting the presence of naphthenic acids in the filtered wastewater.

2. The method of claim 1, wherein fluorescence of the filtered wastewater is measured.

3. The method of claim 2, wherein the measured fluorescence is correlated with concentration of the naphthenic acids in the filtered wastewater using a calibration curve.

4. The method of claim 1, wherein the filter comprises two filters in series, and wherein the fluorescence sensor is between the two filters in series.

5. The method of claim 1, wherein the filter comprises three or more filters in series.

6. The method of claim 1, wherein the filtered wastewater has fluorescence emission is at a wavelength of from about 325 nm to about 355 nm.

7. The method of claim 1, wherein the filtered wastewater is exposed to an excitation wavelength of from about 220 nm to about 300 nm.

8. The method of claim 1, wherein the filter comprises activated carbon.

9. The method of claim 1, wherein the filter consists essentially of activated carbon.

10. The method of claim 1, wherein the method is a continuous method.

11. The method of claim 1, wherein the wastewater is an oil refinery effluent.

12. The method of claim 1, wherein the naphthenic acids comprise at least one phenol.

13. The method of claim 1, wherein the naphthenic acids comprise at least one aromatic carboxylic acid.

14. The method of claim 1, wherein the filtered wastewater is at a temperature of from about 20° C. to about 100° C.

15. A method of monitoring naphthenic acids in wastewater, the method comprising:
filtering wastewater comprising naphthenic acids through a filter having a fluorescence sensor embedded therein and capable of removing naphthenic acids from the wastewater; and
contacting the filtered wastewater with a fluorescence sensor capable of detecting chromophoric components of naphthenic acids and detecting the presence of naphthenic, acids in the filtered wastewater.

16. The method of claim 15, wherein fluorescence of the filtered wastewater is measured.

17. The method of claim 16, wherein the measured fluorescence is correlated with concentration of the naphthenic acids in the filtered wastewater using a calibration curve.

18. The method of claim 15, wherein the filter comprises activated carbon.

19. The method of claim 15, wherein fluorescence of the wastewater is measured at more than one location in the filter.

20. The method of claim 15, wherein the fluorescence sensor is present at a filter inlet and a second fluorescence sensor is present at a filter outlet.
